(19) United States
(12) Patent Application Publication
(10) Pub. No.: US 2015/0004704 A1
(43) Pub. Date: Jan. 1, 2015

(54) COMPOSITIONS AND METHODS FOR INCREASING OIL CONTENT IN ALGAE

(71) Applicant: A.B. Seeds Ltd., Lod (IL)

(72) Inventor: Rudy MAOR, Rechovot (IL)

(21) Appl. No.: 14/318,778

(22) Filed: Jun. 30, 2014

Related U.S. Application Data
(63) Continuation of application No. 13/262,637, filed on Oct. 2, 2011, now abandoned.
(60) Provisional application No. 61/165,912, filed on Apr. 2, 2009.

(51) Int. Cl. C12N 15/82

(52) U.S. Cl. CPC C12N 15/8247; C12N 15/8291
USPC 435/471; 435/257.2

(57) ABSTRACT

The present invention provides methods and compositions for increasing oil content in algae. More particularly, the present invention relates to enhancement of phytohormone activity in algae, by genetic transformation thereof or by supplementing the algal growth medium with phytohormones, to maximize the production of oil within the algae cells.

### RELATED APPLICATIONS

This application is a continuation of U.S. patent application Ser. No. 13/262,637 filed Oct. 2, 2011, which is a National Phase of PCT Patent Application No. PCT/IL2010/000247 having International filing date of Mar. 24, 2010, which claims the benefit of priority of U.S. Provisional Patent Application No. 61/165,912 filed Apr. 2, 2009. The contents of the above applications are all incorporated by reference as if fully set forth herein in their entirety.

### FIELD AND BACKGROUND OF THE INVENTION

The invention relates to methods and compositions for increasing oil content in algae. More particularly, the present invention relates to enhancement of phytohormone activity in algae, by genetic transformation thereof or by supplementing the algal growth medium with phytohormones, to maximize the production of oil within the algae cells.

Microalgae are photosynthetic microorganisms that can be used as a source for several types of biofuels with the most studied one being biodiesel. Algae are fast-growing organisms with naturally high oil content that can potentially be extracted year-round. They can be grown in non-arable areas and thus do not compete with food crops on land. Algae are believed to be at least 30-fold more productive than the best oil-producing land plants and therefore provide the most promising long-term solution for sustainable biofuels production. The huge potential of algae as a feedstock for biodiesel production was the main focus of the Aquatic Species Program (ASP) that was funded by the U.S. Department Of Energy (DOE) between 1978 to 1996.

Algal biotechnology has made considerable advances in recent years including the development of methods for genetic transformation and sequencing the genomes of several algal species. As lipids are the substrates for biodiesel production, significant efforts have been made to increase algal lipid content. These include cloning the ACC1 gene encoding the acetyl-coenzyme A carboxylase (ACCase) enzyme from the diatom Cyclotella cryptica (Sheehan J, Dunahay T, Benemann J, Roessler P: A Look Back at the U.S. Department of Energy's Aquatic Species Program: Biodiesel from Algae; Close-Out Report. Aqua KE Government Documents 1998). Attempts were made to enhance the lipid content of C. crypticafrom by increasing the ACC1 gene copy number. Although an increase of two to three folds in ACCase activity was noted in transgenic lines, no increase in lipid levels was detected.

Nowadays, interest in biodiesel from algae is rapidly growing as the biofuel industry is shifting towards using second generation feedstocks that do not compete with food crops on land and will not contribute to soaring crop prices and the food vs. fuel debate. Accordingly, there is an unmet need to efficiently increase oil content in algae.

### SUMMARY OF THE INVENTION

The present invention provides compositions and methods for enhancing the oil content in algae. The invention is based in part on the finding that enhanced phytohormone-induced activity in algae, achieved by genetic transformation or supplementing algal growth medium, can increase their oil content, thus increasing their biodiesel productivity.

Provided herein is a transgenic alga, comprising an exogenous transgene, wherein expression of the transgene results in enhanced phytohormone activity in the transgenic alga, and wherein the enhanced phytohormone activity results in increased oil content in the transgenic alga as compared to a corresponding wild type alga. According to some embodiments the enhanced phytohormone activity is of a phytohormone selected from the group consisting of Auxin, Cytokinin, Abscisic acid (ABA) and gibberellic acid (GA₃). According to some embodiments the Auxin is selected from the group consisting of indole-3-acetic acid (IAA), 1-naphthaleneacetic acid (NAA), 2, 4-dichlorophenoxyacetic acid (2,4-D). According to some embodiments the Cytokinin is selected from the group consisting of kinetin, zeatin and 6-benzylaminopurine (6-BAP).

According to some embodiments the transgenic alga is selected from the group consisting of Green Algae and Diatoms. According to some embodiments the Green algae is selected from the group consisting of Chlamydomonas reinhardtii, Chlorella vulgaris and Haematococcus pluvialis. According to some embodiments the Diatom is Phaeodactylum tricornutum.

According to some embodiments the exogenous transgene comprises SEQ ID NO. 1, a fragment thereof, a nucleic acid sequence capable of hybridizing thereto under stringent conditions, or sequences having at least 80% identity thereto; and the phytohormone which activity is enhanced is Cytokynin. According to some embodiments the exogenous transgene comprises a polynucleotide encoding a polypeptide having at least 80% sequence identity to a polypeptide having an amino acid sequence of SEQ ID NO: 4, wherein said encoded polypeptide is a functional homolog of said polypeptide having an amino acid sequence of SEQ ID NO: 4; and the phytohormone which activity is enhanced is Cytokynin.

According to some embodiments the exogenous transgene is introduced into the alga within a genetic construct further comprising a constitutive promoter or an inducible promoter. According to some embodiments the constitutive promoter is pGenD.

Also provided in another aspect of the invention, is a method for generating an alga having increased oil content as compared to a control alga, the method comprising enhancing the phytohormone activity of said alga. According to some embodiments the enhanced phytohormone activity is of a phytohormone selected from the group consisting of Auxin, Cytokinin, Abscisic acid (ABA) and gibberellic acid (GA₃). According to some embodiments the Auxin is selected from the group consisting of indole-3-acetic acid (IAA), 1-naphthaleneacetic acid (NAA), 2,4-dichlorophenoxyacetic acid (2,4-D). According to some embodiments the Cytokinin is selected from the group consisting of kinetin, zeatin and 6-benzylaminopurine (6-BAP).

According to some embodiments the enhanced phytohormone activity is achieved by transgenic modification of the alga. According to further embodiments the transgenic modification comprises incorporation of an exogenous transgene into said alga, and expression of said transgene results in enhanced phytohormone activity. According to further embodiments the exogenous transgene comprises SEQ ID NO. 1, a fragment thereof, a nucleic acid sequence capable of hybridizing thereto under stringent conditions, or sequences having at least 80% identity thereto; and the phytohormone which activity is enhanced is Cytokynin. According to further embodiments the exogenous transgene comprises a polynucleotide encoding a polypeptide having at least 80% sequence identity to a polypeptide having an amino acid sequence of SEQ ID NO: 4, wherein said encoded polypeptide is a functional homolog of said polypeptide having an amino acid sequence of SEQ ID NO: 4; and the phytohormone which activity is enhanced is Cytokynin. According to some embodiments the exogenous transgene is introduced into the alga within a genetic construct further comprising a constitutive promoter or an inducible promoter. According to some embodiments the constitutive promoter is pGenD. According to some embodiments the construct further comprises a selectable marker. According to some embodiments the selectable marker is aminoglycoside-O-phosphotransferase. According to some embodiments the selectable marker is paromomycin.

According to additional embodiments the expression of the transgene in the transgenic alga is identified by a method comprising RT-PCR. According to some embodiments the RT-PCR comprises use of any or both of a forward primer and a reverse primer. According to further embodiments the forward primer comprises SEQ ID NO. 2 and the reverse primer comprises SEQ ID NO. 3.

According to some embodiments the enhanced phytohormone activity, resulting in increased oil content, is achieved by application of phytohormone to the growth medium of the alga. According to some embodiments the alga is selected from the group consisting of Green Algae and Diatoms. According to some embodiments the Green Algae is selected from the group consisting of Chlamydomonas reinhardti and Haematococcus pluvialis, and according to some embodiments the Diatom is Phaeodactylum tricornutum.

These and other embodiments of the present invention will become apparent in conjunction with the description and claims that follow.

### BRIEF DESCRIPTION OF THE DRAWINGS

FIG. 1: Oil content in Chlamydomonas reinhardtii a week after addition of phytohormones to growth medium. The y-axis represents the oil content as normalized against the average of the two controls to which no supplemental phytohormones were added.

I: IAA 0.5 mg/liter
II: IAA 1.0 mg/liter
III: NAA 0.5 mg/liter
IV: NAA 1.0 mg/liter
V: 6-BAP 0.05 mg/liter
VI: 6-BAP 0.1 mg/liter
VII: Kinetin 0.05 mg/liter
VIII: Kinetin 0.1 mg/liter

FIGS. 2A-C: Oil content in algal species a week after addition of various phytohormones to growth medium (IAA: 1.0 mg/liter, NAA: 1.0 mg/liter, 2,4-D: 1.0 mg/liter, 6-BAP: 0.1 mg/liter, and Kinetin: 0.1 mg/liter). The y-axis represents oil content as normalized against the average of the two controls to which no phytohormones were added.

FIG. 2A: Chlamydomonas reinhardtii;
FIG. 2B: Phaeodactylum tricornutum (Pt);
FIG. 2C: Haematococcus pluvialis (Hp).
FIG. 3: Expression of IPT (isopentenyltransferase) gene (SEQ ID NO: 1) in transgenic clones of Chlamydomonas reinhardtii as measured by qRT-PCR.

The y-axis represents 40-Ct values for each clone (the Ct signal was inverted by subtracting the normalized value from an arbitrarily chosen value of 40, such that low values of normalized-inverted Ct represent low abundance or expression levels of the gene).

FIG. 4: Relative increase in oil content in algal clones obtained by transformation with IPT gene (SEQ ID NO: 1), a week after the beginning of cultivation. The y-axis represents oil content as normalized against the corresponding control alga.

FIG. 4A: Chlamydomonas reinhardti (clones IPT3, IPT21, IPT22)
Analysis was performed when alga were grown in volumes of 10 ml (black bars) 50 ml (white bars) and 500 ml (checkered bars) medium.
FIG. 4B: Chlorella vulgaris (grown in 50 ml medium) (clones CvIPT 5, CvIPT 6, CvIPT 31)

FIG. 5: Absolute oil content in clones of Chlamydomonas reinhardti, obtained by transformation with IPT gene (SEQ ID NO: 1), presented as percent of dry weight (y-axis). Analysis was performed by the gravimetric method. The experiments were performed three times (white bars, black bars and vertical stripes).

### DETAILED DESCRIPTION OF THE INVENTION

The present invention provides compositions and methods for enhancing the oil content in plants. The invention is based in part on the finding that elevated levels of phytohormones (plant hormones) can increase the oil content in algae, thus increasing their biodiesel productivity. The elevated levels may be obtained by genetic transformation of algae or by supplementing the algal growth medium with phytohormones.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

### 1. GENERAL DEFINITIONS

About

As used herein, the term “about” refers to +/−10%.

Algae

Algae include organisms that belong to the phyla Chlorophyta (green algae), Eustigmatophyceae (eustigmatophytes), Phaeophyceae (brown algae), Bacillariophyta (diatoms), Xanthophyceae (yellow-green algae), Haptophyceae (prymnesiophytes), Chrysophyceae (golden algae), Rhodophyta (red algae) and Cyanobacteria, (blue-green algae). Specific examples of algae include, but are not limited to, Neochloris oleoabundans, Scenedesmus dimorphus, Euglena gracilis, Phaeodactylum tricornutum, Pleurochrysis carterae, Tetraselmis chui, Tetraselmis suecica, Isochrysis galbana, Nannochloropsis sauna, Botryococcus braunii, Dunaliella tertiolecta, Nannochloris sp., Spirulina sp., Chlorella sp., Crypthecodinium cohnii, Cylindrotheca sp., Dunaliella primolecta, Monallanthus salina, Nitzschia sp., Schizochytrium sp. and Tetraselmis sueica.

Auxotrophy

As used herein, auxotrophy is the inability of an organism to synthesize a particular organic compound required for its growth.

Ct

Ct signals represent the first cycle of PCR where amplification crosses a threshold (cycle threshold) of fluorescence. Accordingly, low values of Ct represent high abundance or expression levels of the amplified sequence. The PCR Ct signal may be normalized and then inverted such that low normalized-inverted Cts represent low abundance or low expression levels of the amplified sequence.

Complement

“Complement” or “complementary” as used herein to refer to a nucleic acid may mean Watson-Crick or Hoogsteen base pairing between nucleotides or nucleotide analogs of nucleic acid molecules. A full complement or fully complementary means 100% complementary base pairing between nucleotides or nucleotide analogs of nucleic acid molecules. In some embodiments, the complementary sequence has a reverse orientation (5′-3′).

Constitutive Promoter

As used herein, a constitutive promoter is an unregulated promoter that allows for continual transcription of its associated gene.

Enhanced Phytohormone Activity

As used herein, enhanced phytohormone activity is when the phytohormone activity is at least 5%, preferably at least 20%, particularly preferably at least 40%, very particularly preferably at least 60%, most preferably at least 85% enhanced under otherwise identical conditions in comparison with a starting alga which has not been subjected to the method according to the invention.

Expression Vector

The expression vector or cassette typically comprises an encoding sequence operably linked (i.e., under regulatory control of) to appropriate inducible or constitutive regulatory sequences that allow for the expression of polypeptide. The expression vector can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucleic Acids Res. 12: 8711-8721, Klee (1985) Bio/Technology 3: 637-642, for dicotyledonous plants. A number of expression vectors suitable for stable transformation of algal cells or for the establishment of transgenic algae have been described including those described in Hallmann, A (2007) Transgenic Plant Journal 1(1):81-98

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter, a transcription initiation start site, an RNA processing signal (such as intron splice sites), a transcription termination site, and/or a polyadenylation signal.

Fragment

“Fragment” is used herein to indicate a non-full length part of a nucleic acid or polypeptide. Thus, a fragment is itself also a nucleic acid or polypeptide, respectively. Generally, fragments will be ten or more nucleotides in length.

Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

Genetic Construct

As used herein, a genetic construct is an artificially constructed segment of nucleic acid that is to be transplanted into a target tissue or cell. It often contains a DNA insert, which contains the gene sequence encoding a protein of interest that has been subcloned into a vector, which contains promoter(s) for expression in the organism.

Gravimetric Analysis

As used herein, gravimetric analysis describes a set of methods in analytical chemistry for the quantitative determination of an analyte based on the mass of a solid.

In most cases, the analyte must first be converted to a solid by precipitation with an appropriate reagent. The precipitate can then be collected by filtration, washed, dried to remove traces of moisture from the solution, and weighed. The amount of analyte in the original sample can then be calculated from the mass of the precipitate and its chemical composition.

Gravimetric method provides for exceedingly precise analysis. Gravimetry provides very little room for instrumental error and does not require a series of standards for calculation of an unknown value. Due to its high degree of accuracy, gravimetric analysis can also be used to calibrate other instruments in lieu of reference standards.

Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

Increased Oil Content

As used herein, increased oil content is when the oil content is at least 5%, preferably at least 20%, particularly preferably at least 40%, very particularly preferably at least 60%, most preferably at least 85% increased under otherwise identical conditions in comparison with a starting alga which has not been subjected to the method according to the invention.

Inducible Promoter

As used herein, the term “inducible promoter” means a promoter that is turned on by the presence or absence of a particular stimulus that increases promoter activity directly or indirectly. Some non-limiting examples of such stimuli include heat, light, developmental regulatory factors, wounding, hormones, and chemicals, e.g., small molecules.

Modified Expression (or: Modulated Expression)

As used herein, modified expression in reference to a nucleic acid sequence indicates that the pattern of expression in, e.g., a transgenic alga, is different from the expression pattern in a wild-type alga or a reference alga of the same species. The pattern of expression may also be compared with a reference expression pattern in a wild-type alga of the same species. For example, the nucleic acid is expressed in a cell or tissue type other than a cell or tissue type in which the sequence is expressed in the wild-type alga, or by expression at a time other than at the time the sequence is expressed in the wild-type alga, or by a response to different inducible agents, such as hormones or environmental signals, or at different expression levels (either higher or lower) compared with those found in a wild-type alga. The term also refers to modified expression patterns that are produced by lowering the levels of expression to below the detection level or completely abolishing expression. The resulting expression pattern can be transient or stable, constitutive or inducible.

Nucleic Acid

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methyl phosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated herein by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005), Soutschek et al., Nature 432:173-178 (2004), and U.S. Patent Publication No. 20050107325, which are incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver and kidney. Mixtures of naturally occurring nucleic acids and analogs may be made; alternatively, mixtures of different nucleic acid analogs, and mixtures of naturally occurring nucleic acids and analogs may be made.

Overexpression

The term “overexpression” as used herein refers to a greater expression level of a gene, compared to expression in the wild-type plant, at any developmental or temporal stage for the gene. Overexpression can occur when, for example, the genes encoding one or more transcription factors are under the control of a strong expression signal. Overexpression results in a greater than normal production, or “overproduction” of a gene product.

Phytohormones (Also: Plant Hormones)

Phytohormones are biologically active organic compounds which in minute quantities function to promote or modify plant (and algal) growth. Phytohormones are classified as auxin, cytokinin, gibberellin and abscisic acid types as relating to different growth regulating activities.

Polypeptide

As used herein, the term “polypeptide” means an unbranched chain of amino acid residues that are covalently linked by an amide linkage between the carboxyl group of one amino acid and the amino group of another. The term polypeptide can encompass whole proteins (i.e. a functional protein encoded by a particular gene), as well as fragments of proteins. Of particular interest are polypeptides which represent whole proteins or a sufficient portion of the entire protein to impart the relevant biological activity of the protein. The term “protein” also includes molecules consisting of one or more polypeptide chains. Thus, a polypeptide for use in constructs of the present invention may also constitute an entire gene product, but only a portion of a functional oligomeric protein having multiple polypeptide chains. The invention utilizes polynucleotides that encode polypeptides identified from a microbial source. The encoded polypeptides may be the complete protein encoded by an identified microbial gene, or may be fragments of the encoded protein. Preferably, polynucleotides utilized herein encode polypeptides constituting a substantial portion of the complete protein, and more preferentially, constituting a sufficient portion of the complete protein to provide the relevant biological activity.

Probe

“Probe” as used herein means an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur even under the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Promoter

A sequence of nucleotides from which transcription may be initiated of DNA operably linked downstream (i.e. in the 3′ direction on the sense strand of double-stranded DNA). A promoter “drives” transcription of an operably linked sequence. “Operably linked” means joined as part of the same nucleic acid molecule, suitably positioned and oriented for transcription to be initiated from the promoter.

Promoters are expressed in many, if not all, cell types of many plants. Promoters including those that are developmentally regulated or inducible may be used. For example, if it is necessary to silence the target gene specifically in a particular cell type a construct may be assembled with a promoter that drives transcription only in that cell type. Similarly, if the target gene is to be silenced following a defined external stimulus the construct may incorporate a promoter that is be activated specifically by that stimulus. Promoters that are both tissue specific and inducible by specific stimuli may be used.

A variety of plant gene promoters that regulate gene expression in response to environmental, hormonal, chemical, developmental signals can be used in plants in a tissue-active manner. Choice of a promoter is based largely on the phenotype of interest and is determined by such factors as tissue, inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic organism of interest.

Second Generation Feedstocks

Feedstocks refer to the crops or products that can be used as or converted into biofuels and bioenergy. Each feedstock has advantages and disadvantages in terms of how much usable material they yield, where they can grow and how energy and water-intensive they are. As used herein, second generation feedstocks refers broadly to crops that have high potential yields of biofuels, but that are not widely cultivated, or not cultivated as an energy crop.

Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probe molecules complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times stronger than background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Transgene

A transgene is a gene or genetic material that has been transferred naturally or by any of a number of genetic engineering techniques from one organism to another. The non-native segment of DNA may retain the ability to produce RNA or protein in the transgenic organism, or it may alter the normal function of the transgenic organism's genetic code.

The transgene can describe any DNA sequence, regardless of whether it contains a gene coding sequence or it has been artificially constructed, which has been introduced into an organism or vector construct in which it was previously not found.

A transgene can be either a cDNA (complementary DNA) segment, which is a copy of mRNA (messenger RNA), or the gene itself residing in its original region of genomic DNA.

Transgenic Alga

A transgenic alga as used herein refers to an alga that contains genetic material not found in a wild-type alga of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the alga by human manipulation, but any method can be used as one of skill in the art recognizes. A transgenic alga may contain an expression vector or cassette.

After transformed algae are selected and grown to maturity, those algae showing a modified trait are identified.

Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Wild Type

As used herein, “wild type” is the most common form or phenotype in nature or in a natural breeding population. Wild-type organisms serve as the original parent strain before a deliberate change is introduced. They are used as a reference when comparing naturally occurring genotypes and phenotypes of a given species against those of deliberately changed counterparts.

### 2. ALGAL TECHNOLOGY

Although there are thousands of species of known naturally occurring algae, any one may be used for biodiesel production according to the embodiments of the invention. The skilled artisan will realize that different algal strains will exhibit different growth and oil productivity and that under different conditions the system may contain a single strain of algae or a mixture of strains with different properties, or strains of algae plus symbiotic bacteria. The algal species used may be optimized for geographic location, temperature sensitivity, light intensity, pH sensitivity, salinity, water quality, nutrient availability, seasonal differences in temperature or light, the desired end products to be obtained from the algae and a variety of other factors.

### 2.1 Genetic Modification of Algae

The genetic modification of algae for specific product outputs is relatively straight forward using techniques well known in the art. In certain embodiments, algae of use to produce biodiesel may be genetically engineered (transgenic) to contain one or more isolated nucleic acid sequences that enhance oil production or provide other characteristics of use for algal culture, growth, harvesting or use. Methods of stably transforming algal species and compositions comprising isolated nucleic acids of use are well known in the art and any such methods and compositions may be used in the practice of the present invention. Exemplary transformation methods of use may include microprojectile bombardment, electroporation, protoplast fusion, PEG-mediated transformation, DNA-coated silicon carbide whiskers or use of viral mediated transformation (see, e.g., Sanford et al., 1993, Meth. Enzymol. 217:483-509; Dunahay et al., 1997, Meth. Molec. Biol. 62:503-9, incorporated herein by reference).

U.S. Pat. No. 5,661,017 discloses methods for algal transformation of chlorophyll C-containing algae, such as the Bacillariophyceae, Chrysophyceae, Phaeophyceae, Xanthophyceae, Raphidophyceae, Prymnesiophyceae, Cryptophyceae, Cyclotella, Navicula, Cylindrotheca, Phaeodactylum, Amphora, Chaetoceros, Nitzschia or Thalassiosira.

The particular choice of a transformation technology will be determined by its efficiency to transform certain algal species as well as the experience and preference of the person practicing the invention with a particular methodology of choice. It will be apparent to the skilled person that the particular choice of a transformation system to introduce nucleic acid into algal cells is not essential to or a limitation of the invention, nor is the choice of technique for algal growth.

When appropriate, the gene(s) for enhanced expression may be optimized for expression in the transformed alga. That is, the genes can be synthesized using preferred codons corresponding to the alga of interest. Methods are available in the art for synthesizing preferred genes. Degeneracy of the genetic code provides the possibility to substitute at least one base of the protein encoding sequence of a gene with a different base without causing the amino acid sequence of the polypeptide produced from the gene to be changed. Hence, the DNA utilized in the present invention may also have any base sequence that has been changed from SEQ ID NO: 1 by substitution in accordance with degeneracy of the genetic code.

Following transformation, algae are preferably selected using a dominant selectable marker incorporated into the transformation vector. Typically, such a marker will confer antibiotic or other resistance on the transformed algae and selection of transformants can be accomplished by exposing the algae to appropriate concentrations of the selectable marker to select for transformed algae. Selectable markers of use may include, paromomycin, neomycin phosphotransferase, aminoglycoside phosphotransferase, aminoglycoside-O-phosphotransferase, aminoglycoside acetyltransferase, chloramphenicol acetyl transferase, hygromycin B phosphotransferase, bleomycin binding protein, phosphinothricin acetyltransferase, bromoxynil nitrilase, glyphosate-resistant 5-enolpyruvylshikimate-3-phosphate synthase, cryptopleurine-resistant ribosomal protein S 14, emetine-resistant ribosomal protein S 14, sulfonylurea-resistant acetolactate synthase, imidazolinone-resistant acetolactate synthase, streptomycin-resistant 16S ribosomal RNA, spectinomycin-resistant 16S ribosomal RNA, erythromycin-resistant 23 S ribosomal RNA or methyl benzimidazole-resistant tubulin. Regulatory nucleic acid sequences to enhance expression of a transgene are known, such as C. cryptica acetyl-CoA carboxylase 5′-untranslated regulatory control sequence, a C. cryptica acetyl-CoA carboxylase 3′-untranslated regulatory control sequence, and combinations thereof.

Additional possible selection is via auxotroph mutants. The selectable marker is then a biosynthetic gene allowing the mutant to grow in the absence of the substance they are auxotroph to. This includes the ability to synthesize amino acids, vitamins and other essential co-factors. A specific example is the ARG7 locus that can rescue the arg2 mutation in C. reinhardtii (Debuchy et al. (1989) EMBO J. 8, 2803-2809) and allow cells to grow in the absence of the amino acid arginine.

After transformed algae are selected, those with a modified trait are identified. The modified trait can be enhanced oil content.

To confirm that the modified trait is due to changes in expression levels or activity of nucleic acid sequences of the invention, expression can be analyzed by using Northern blots, RT-PCR or microarrays. Expression levels may be assessed by determining the level of a gene product by any known in the art including, but not limited to determining the level of the RNA and protein encoded by a particular target gene. For genes that encode proteins, expression levels may be determined, for example, by quantifying the amount of the protein present in the algal cells. Alternatively, if the target gene encodes protein that has a known measurable activity, then activity levels may be measured to assess expression levels.

### 2.2 Separation of Algae and Extraction of Oil

In various embodiments, algae may be separated from the medium, and various algal components, such as oil, may be extracted using any method known in the art. For example, algae may be partially separated from the medium using a standing whirlpool circulation, harvesting vortex and/or sipper tubes, as discussed below. Alternatively, industrial scale commercial centrifuges of large volume capacity may be used to supplement or in place of other separation methods. Such centrifuges may be obtained from known commercial sources (e.g., Cimbria Sket or IBG Monforts, Germany; Alfa Laval AJS, Denmark). Centrifugation, sedimentation and/or filtering may also be of use to purify oil from other algal components. Separation of algae from the aqueous medium may be facilitated by addition of flocculants, such as clay (e.g., particle size less than 2 microns), aluminum sulfate or polyacrylamide. In the presence of flocculants, algae may be separated by simple gravitational settling, or may be more easily separated by centrifugation.

The skilled artisan will realize that any method known in the art for separating cells, such as algae, from liquid medium may be utilized. U.S. Pat. No. 6,524,486, incorporated herein by reference, discloses a tangential flow filter device and apparatus for partially separating algae from an aqueous medium Other published methods for algal separation and/or extraction may also be used. (See, e.g., Rose et al., Water Science and Technology 1992, 25:319-327; Smith et al., Northwest Science, 1968, 42:165-171; Moulton et al., Hydrobiologia 1990, 204/205:401-408; Borowitzka et al., Bulletin of Marine Science, 1990, 47:244-252; Honeycutt, Biotechnology and Bioengineering Symp. 1983, 13:567-575).

In various embodiments, algae maybe disrupted to facilitate separation of oil and other components. Any method known for cell disruption may be utilized, such as ultrasonication, French press, osmotic shock, mechanical shear force, cold press, thermal shock, rotor-stator disruptors, valve-type processors, fixed geometry processors, nitrogen decompression or any other known method. High capacity commercial cell disruptors may be purchased from known sources. (E.g., GEA Niro Inc., Columbia, Md.; Constant Systems Ltd., Daventry, England; Microfluidics, Newton, Mass.) Methods for rupturing microalgae in aqueous suspension are disclosed, for example, in U.S. Pat. No. 6,000,551, incorporated herein by reference.

### 3. PHYTOHORMONES

Phytohormones (also: plant hormones) are small organic molecules that affect diverse developmental processes. Virtually every aspect of plant development from embryogenesis to senescence is controlled by phtyohormone concentration and location including regulation of cell division, cell elongation, cell differentiation and cell death. Phytohormones are classified as auxin, cytokinin, gibberellin and abscisic acid types as relating to different plant growth regulating activities. Auxins affect cell division and growth, cytokinins affect the organization of dividing cells, gibberellins promote the growth of seedlings and have other specific activities of the auxin type, abscisic acids exhibit abscission-accelerating activity Important beneficial effects derive from the use of phytohormones as plant growth regulators in agricultural applications, which include increased resistance of plants to frost, fungal and insect attack, increased uptake of inorganic constituents from the soil, increased photosynthetic power; stabilization of chlorophyll, reduction in fertilizer requirements, increased crop yield, herbicidal activity; and the like.

### EXAMPLES

### Example 1

### Material and Methods

### 1. Algal Material and Growth Conditions:

All algal strains were obtained from University of Texas algae collection, Austin

TX, and were grown at 22° C. under constant white light at an intensity of 175 micromole photon m⁻² s−1. Chlamydomonas reinhardtii (UTEX strain 90), Chlorella vulgaris (UTEX 2714) and Haematococcus pluvialis (UTEX strain 2505) were grown in TAP medium {Harris E H: The Chlamydomonas sourcebook: A comprehensive guide to biology and laboratory use. New-York: Academic Press; 1989}. Phaeodactylum tricornutum (UTEX strain 642) was grown in a modified LDM medium {Lewin, R. A., 1959. The isolation of algae. Rev. algol. no. 3: 182-197}.

### 2. Plant Hormones:

All plant hormones were obtained from Sigma-Aldrich.
Indole-3-acetic acid (IAA) sodium salt—15148-2G, 1 mg/ml stock solution prepared in sterile double-deionized water (ddH₂O).
α-Naphthaleneacetic acid (NAA) solution, 1 mg/ml. N1641.
2,4-dichlorophenoxy acetic acid (2,4-D) sodium salt monohydrate—D6679-5G. 1 mg/ml stock solution in ddH₂O.
6-Benzylaminopurine (6-BAP)—B3408-100 mg. 1 mg/ml stock solution prepared in 0.1N NaOH.
Kinetin solution, 1 mg/ml. K3253.

### Example 2

### Oil Biosynthesis in Chlamydomonas reinhardtii with Hormone Supplements

Algae were grown to a concentration of 10⁵ cells/ml and divided into 10×250-ml flasks, each containing 50 ml TAP medium with the following hormone supplements (all values are final hormone concentrations):

IAA: 0.5 mg/liter and 1 mg/liter,
NAA: 0.5 mg/liter and 1 mg/liter,
6-BAP: 0.05 mg/liter and 0.1 mg/liter, and
Kinetin: 0.05 mg/liter and 0.1 mg/liter.
No supplement was added to Controls 1 and 2.

Oil content was measured, in duplicate, one week after addition of the hormones to the medium, using Nile Red staining. 200 μl of Chlamydomonas reinhardtii cells were placed in a 96-well plate (Costar 3596), mixed with 10 μl of Nile Red solution (N3013, Sigma. 1 mg/ml stock in acetone) and incubated for 15 minutes at room-temperature. Fluorescence was measured using a Thermo Electron Fluroskan Ascent with excitation at 544 nm and emission at 590 nm 200 μl of fresh growth media with 10 μl of Nile Red were used for blank measurements. Fluorescence was normalized against cell density as measured by an EL×808 microplate reader (BIOTEK Instruments Inc) at 450 nm and verified by cell count of selected samples.

The growth rate in each flask was estimated by measuring the OD (optical density, at 450 nm), which represents cell density. For each treatment the normalized oil content was defined as the oil content divided by the cell density. The average of the normalized oil content of the duplicates for each treatment was calculated. The effect of each treatment was determined by the ratio between the average of the normalized oil content and the average of the two control experiments. The results are presented in table 1 below.

TABLE 1
Cell density and oil content in Chlamydomonas reinhardtii
1 week after addition of hormones to growth medium

| | | | | Average | |
|---|---|---|---|---|---|
| Supple- | Cell | Oil | Normalized | normalized | |
| ment | density | content | oil content | oil content | Effect |
| Control 1 | 0.37 | 2.635 | 7.10 | 7.29 | — |
| | 0.36 | 2.678 | 7.48 | | |
| Control 2 | 0.35 | 2.57 | 7.34 | 7.33 | — |
| | 0.34 | 2.511 | 7.32 | | |
| IAA | 0.29 | 2.747 | 9.57 | 10.17 | 1.39 ± 0.11 |
| 0.5 mg/l | 0.26 | 2.748 | 10.78 | | |
| IAA | 0.33 | 3.283 | 9.89 | 10.92 | 1.49 ± 0.20 |
| 1.0 mg/l | 0.30 | 3.635 | 11.96 | | |
| NAA | 0.32 | 2.677 | 8.44 | 8.67 | 1.19 ± 0.04 |
| 0.5 mg/l | 0.29 | 2.59 | 8.90 | | |
| NAA | 0.31 | 3.059 | 9.84 | 10.36 | 1.42 ± 0.10 |
| 1.0 mg/1 | 0.30 | 3.232 | 10.88 | | |
| 6-BAP | 0.29 | 2.709 | 9.28 | 9.41 | 1.29 ± 0.02 |
| 0.05 mg/l | 0.27 | 2.587 | 9.55 | | |
| 6-BAP | 0.28 | 3.04 | 10.74 | 10.65 | 1.46 ± 0.02 |
| 0.1 mg/l | 0.27 | 2.891 | 10.55 | | |
| Kinetin | 0.31 | 3.023 | 9.85 | 10.47 | 1.43 ± 0.12 |
| 0.05 mg/l | 0.28 | 3.071 | 11.09 | | |
| Kinetin | 0.29 | 3.503 | 11.91 | 12.24 | 1.67 ± 0.06 |
| 0.1 mg/l | 0.27 | 3.38 | 12.57 | | |

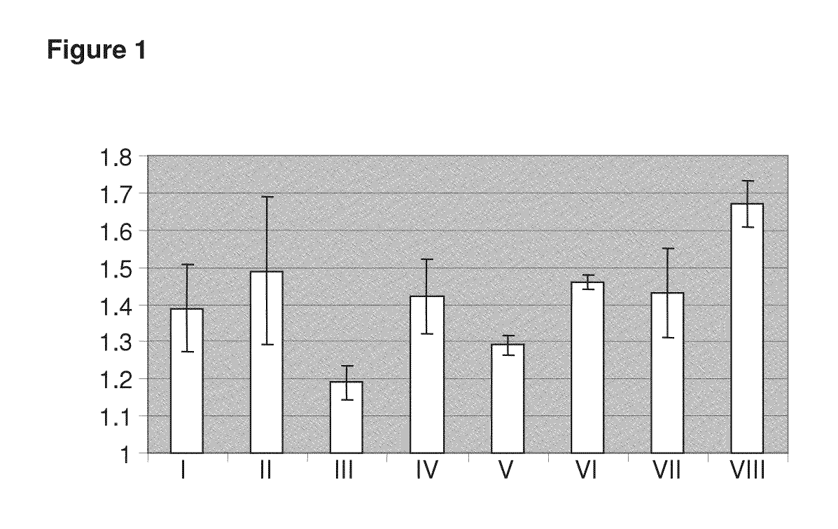

As apparent from table 1 and further indicated in FIG. 1, the maximum effect was found in the kinetin 0.1 mg/liter treatment, with a 67% increase compared to controls.

### Example 3

### Oil Biosynthesis in Chlamydomonas reinhardtii, Phaeodactylum Tricornutum and Haematococcus pluvialis, with Hormone Supplements

Algal cells of Chlamydomonas reinhardtii, Phaeodactylum tricornutum and Haematococcus pluvialis, were each grown to a concentration of 10⁵ cells/ml and divided into 7×250-ml flasks, each containing 50 ml medium with the following supplements (all values are final hormone concentrations):

IAA: 1.0 mg/liter,
NAA: 1.0 mg/liter,
2,4-D: 1.0 mg/liter,
6-BAP: 0.1 mg/liter, and
Kinetin: 0.1 mg/liter.

No supplement was added to Controls 1 and 2.

Oil content was measured in triplicate 1 week after addition of the hormones to the medium, using Nile Red staining: 200 μl of algal cells were placed in a 96-well plate (Costar 3596), mixed with 10 μl of Nile Red solution (N3013, Sigma. 1 mg/ml stock in acetone) and incubated for 10 minutes at room-temperature. Fluorescence was measured using a Thermo Electron Fluroskan Ascent with excitation at 485 nm and emission at 590 nm 200 μl of fresh growth media with 10 μl of Nile Red were used for blank measurements. Fluorescence was normalized against cell density as measured by an EL×808 microplate reader (BIOTEK Instruments Inc) at 630 nm and verified by cell count of selected samples. For each treatment the normalized oil content was defined as the oil content divided by the cell density. The average of the normalized oil content of the triplicates for each treatment was calculated. The effect of each treatment was determined by the ratio between the average of the normalized oil content and the average of the two control experiments.

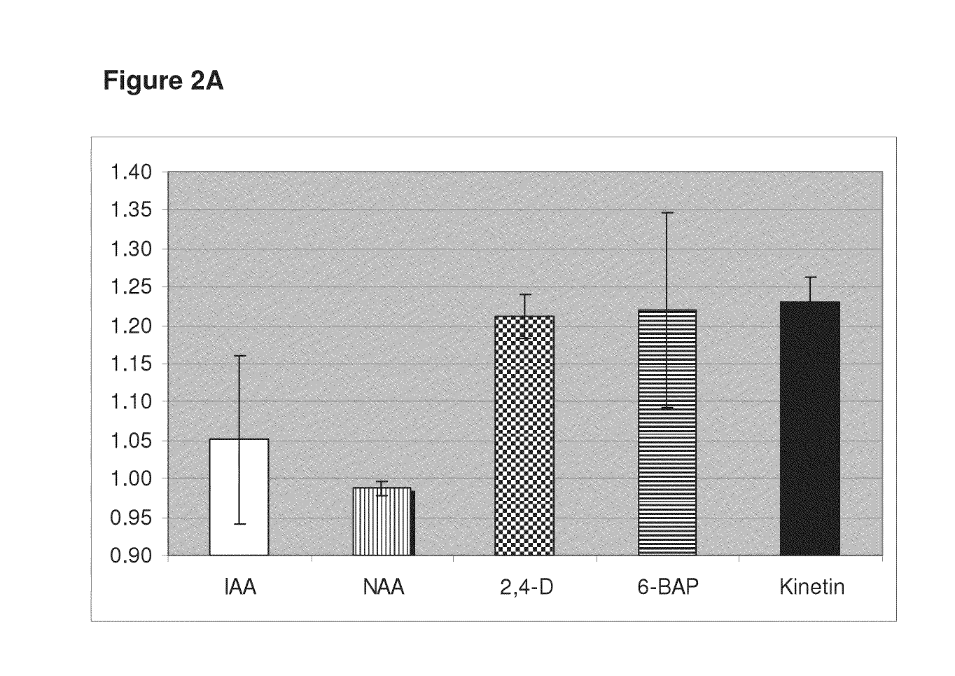

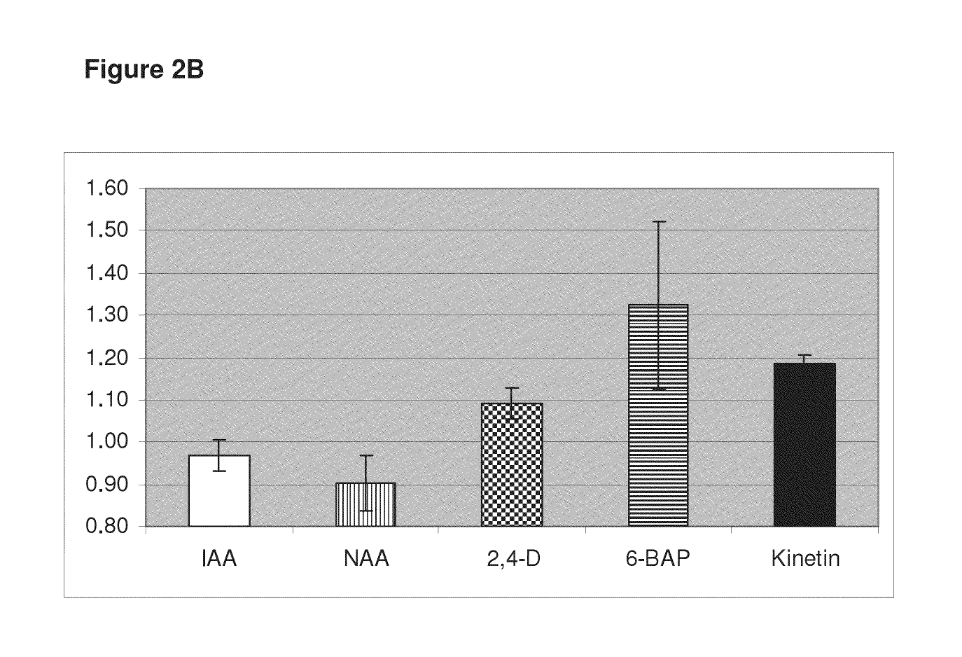

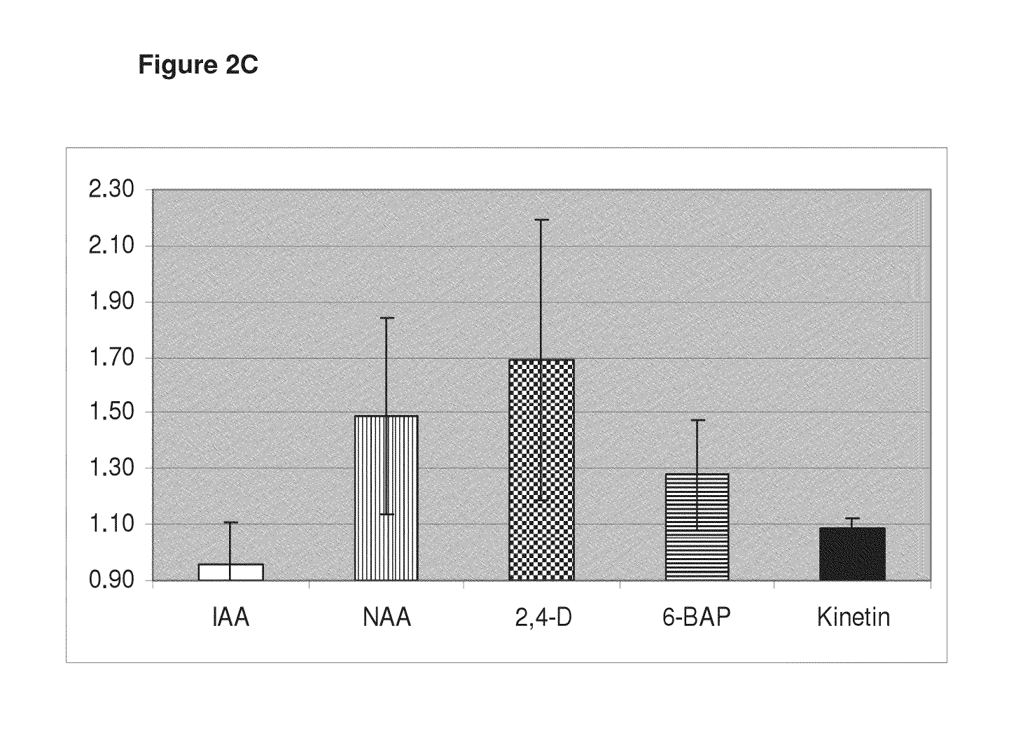

Results are presented in table 2 below, and in FIGS. 2A-2C.

TABLE 2
Effect of addition of hormones to growth medium
on oil content in various algal species

| | Chlamydomonas | Phaeodactylum | Haematococcus |
|---|---|---|---|
| Supplement | reinhardtii | tricornutum | pluvialis |
| IAA 1 mg/l | 1.05 ± 0.11 | 0.97 ± 0.04 | 0.96 ± 0.15 |
| NAA 1 mg/l | 0.99 ± 0.01 | 0.90 ± 0.07 | 1.49 ± 0.35 |
| 2,4-D 1 mg/l | 1.21 ± 0.03 | 1.09 ± 0.04 | 1.69 ± 0.51 |
| 6-BAP 0.1 mg/l | 1.22 ± 0.13 | 1.32 ± 0.20 | 1.28 ± 0.20 |
| Kinetin 0.1 mg/l | 1.23 ± 0.03 | 1.19 ± 0.02 | 1.09 ± 0.03 |

### Example 4

### Oil Content in Algal Lines Transformed with a Cytokinine Biosynthetic Gene

### Example 4.1

### Transformation of Algal Clones

The IPT (isopentenyltransferase) gene (SEQ ID NO: 1) from Agrobacterium tumefaciens (GeneID: 1224196) was synthesized and cloned into the pUC57 vector so that it contains NdeI and EcoRI restriction sites at the 5′ and 3′ ends respectively. The vector was digested with NdeI and EcoRI to release the fragment containing the IPT gene, which was further cloned to the pGenD-Ble vector (N. Fischer & J. D. Rochaix, Molecular General Genomics, 2001, 265:888-894). pGenD-Ble was previously digested with NdeI and EcoRI to release the ble gene. The digested pGenD-Ble vector was separated by gel electrophoresis; the fragment containing the vector minus the BLE gene was excised from the gel and ligated into the IPT gene (SEQ ID NO: 1). The resulting vector containing the pGenD promoter-IPT open reading frame and pGenD terminator was designated pGenD-IPT.
The pGenD-IPT vector was co-transformed into Chlamydomonas Reinhardtii cw15 together with pGenD-Aph following the method described by K Kindle (K. L. Kindle, High frequency nuclear transformation of Chlamydomonas reinhardtii, Proc. Natl. Acad. Sci. U.S.A. 93 (1990) 13689-13693). PgenD-Aph was prepared by introducing the Aph gene (I. Sizova, M. Fuhrmann and P. Hegemann, Gene 277 (2001) 221-229) between the NdeI and EcoRI sites of pGenD-Ble as described above. Cells were selected on TAP medium containing 15 microgram/ml paromomycin.
The pGenD-IPT vector was also co-transformed into Chlorella vulgaris, together with pGenD-Aph using the Biolistic® PDS-1000/He Particle Delivery System (BioRad) following the conditions described by Dawson et al (Current Microbiology, 1997, 35(6) 356-362). Cells were selected on TAP medium containing 150 microgram/ml paromomycin.

### Example 4.2

### Validation of IPT Expression

The expression of the IPT gene (SEQ ID NO: 1) in four transgenic clones of Chlamydomonas Reinhardtii (clones IPT3, IPT21, IPT22) was measured by qRT-PCR.

TABLE 3
Sequence of the IPT gene (SEQ ID NO: 1) and
sequences used for identification of its
expression by qRT-PCR

| | | SEQ |
|---|---|---|
| | | ID |
| | Sequence | NO. |
| IPT gene | CATATGGACCTGCATCTAATTTTCGGTCCA | 1 |
| | ACTTGCACAGGAAAGACGACGACCGCGATA | |
| | GCTCTTGCCCAGCAGACAGGGCTTCCAGTC | |
| | CTTTCGCTTGATCGGGTCCAATGCTGTCCT | |
| | CAACTATCAACCGGAAGCGGACGACCAACA | |
| | GTGGAAGAACTGAAAGGAACGACGCGTCTC | |
| | TACCTTGATGATCGGCCTCTGGTGGAGGGT | |
| | ATCATCGCAGCCAAGCAAGCTCATCATAGG | |
| | CTGATCGAGGAGGTGTATAATCATGAGGCC | |
| | AACGGCGGGCTTATTCTTGAGGGAGGATCC | |
| | ACCTCGTTGCTCAACTGCATGGCGCGAAAC | |
| | AGCTATTGGAGTGCAGATTTTCGTTGGCAT | |
| | ATTATTCGCCACAAGTTACCCGACCAAGAG | |
| | ACCTTCATGAAAGCGGCCAAGGCCAGAGTT | |
| | AAGCAGATGTTGCACCCCGCTGCAGGCCAT | |
| | TCTATTATTCAAGAGTTGGTTTATCTTTGG | |
| | AATGAACCTCGGCTGAGGCCCATTCTGAAA | |
| | GAGATCGATGGATATCGATATGCCATGTTG | |
| | TTTGCTAGCCAGAACCAGATCACGGCAGAT | |
| | ATGCTATTGCAGCTTGACGCAAATATGGAA | |
| | GGTAAGTTGATTAATGGGATCGCTCAGGAG | |
| | TATTTCATCCATGCGCGCCAACAGGAACAG | |
| | AAATTCCCCCAAGTTAACGCAGCCGCTTTC | |
| | GACGGATTCGAAGGTCATCCGTTCGGAATG | |
| | TATTAGGAATTC | |
| Forward primer | TAGGCTGATCGAGGAGGTGT | 2 |
| Reverse primer | CTGCTTAACTCTGGCCTTGG | 3 |

The protocol of the qRT-PCR reaction is as follows:

mRNA—First Strand cDNA with polyT Adaptor:

1. 1 μg total RNA in 4.5 μl double distilled water (DDW).

2. Incubate at 85° C. for 2 min and put on ice for 2 min.

3. Add on ice reaction mixture 5.5 μl:

| | Mix x1 |
|---|---|
| 5 X buffer (Invitrogen) | 2 |
| 0.1M DTT | 1 |
| RNAsin | 0.5 |
| Oligo dT primer (10 μM stock) | 1.0 |
| 10 mM dNTP's | 0.5 |
| RT superscript II (Invitrogen) | 0.5 |

4. Incubate for 1 hr at 42° c.

5. Incubate at 85° C. for 2 min

6. Spin and place on ice.
Final concentration: 100 ng/μl, total of 10 μl.
   Real-Time PCR using SYBR Green

1. Dilute the cDNA from 100 ng/μl to 10 ng/μl

2. Assemble 10 μl SYBR Green (ABI) and 7 μl DDW.

3. Vortex and short spin.

4. Add 1 μl cDNA 10 ng/μl
5. Mix by flipping, vortex and short spin.
6. Divide 18 μl from the mix into each well.
7. Add 1 μl (10 μM stock) Forward primer (SEQ ID NO: 2) 1 μl (10 μM stock) Reverse primer (SEQ ID NO: 3)
8. Short spin the plate and start the reaction.

The reaction program:

stage 1, Reps=1
step 1: Hold @ 95.0 C for 10:00 (MM:SS),

### Stage 2, Reps=42

### Step 1: Hold @ 95.0 C for 0:15 (MM:SS),

### Step 2: Hold @ 60.0 C for 1:00 (MM:SS),

### Dissociation Protocol:

### Stage 3, Reps=1

### Step 1: Hold @ 95.0 C for 0:15 (MM:SS),

### Step 2: Hold @ 60.0 C for 1:00 (MM:SS),

### Step 3: Hold @ 95.0 C for 0:15 (MM:SS).

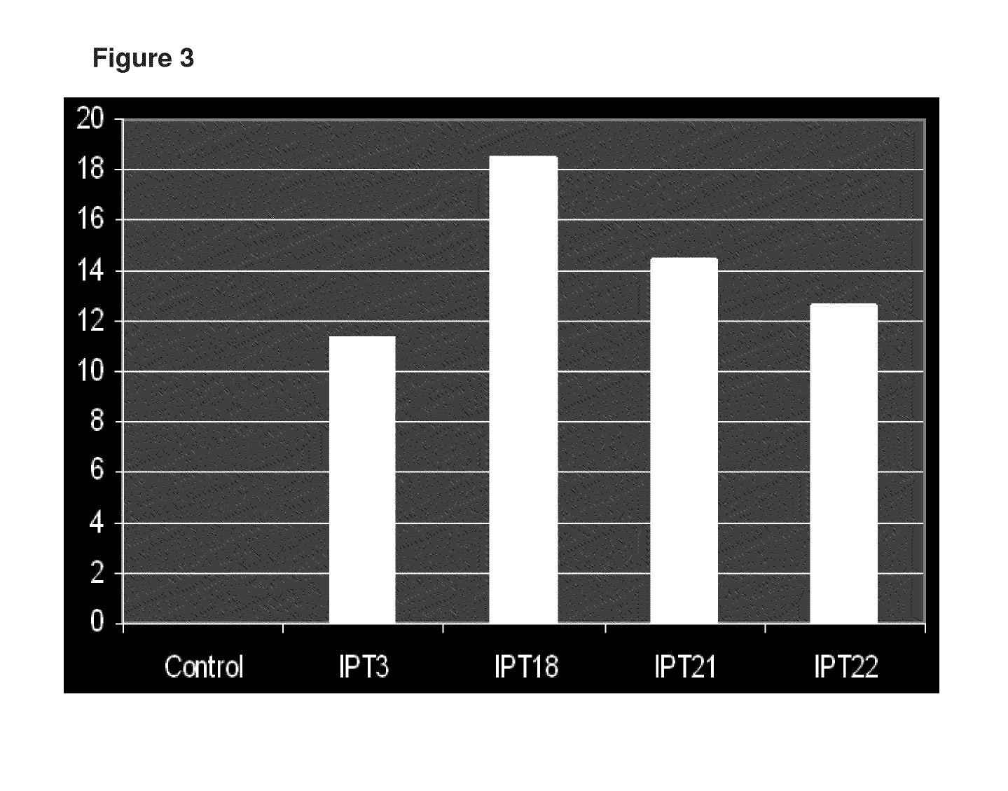

The Ct signal of the qRT-PCR was inverted by subtracting the value from an arbitrarily chosen value of 40, such that low values of the inverted Ct represent low abundance or expression levels of the gene. As indicated in table 4 below and in FIG. 3, no IPT expression was detected in the wild-type control.

TABLE 4
IPT gene (SEQ ID NO: 1) expression (in units of 40 −
Ct) in transgenic clones of Chlamydomonas reinhardtii

| Clone | expression |
|---|---|
| control | 0 |
| IPT3 | 11.3 |
| IPT18 | 18.5 |
| IPT21 | 14.4 |
| IPT22 | 12.6 |

As apparent from table 4 and FIG. 3, the IPT gene (SEQ ID NO: 1) was successfully transformed into the IPT3, IPT18, IPT21 and IPT22 clones of Chlamydomonas reinhardtii.

### Example 4.3

### Oil Content in Algal Clones Transformed with the IPT Gene (SEQ ID NO: 1)

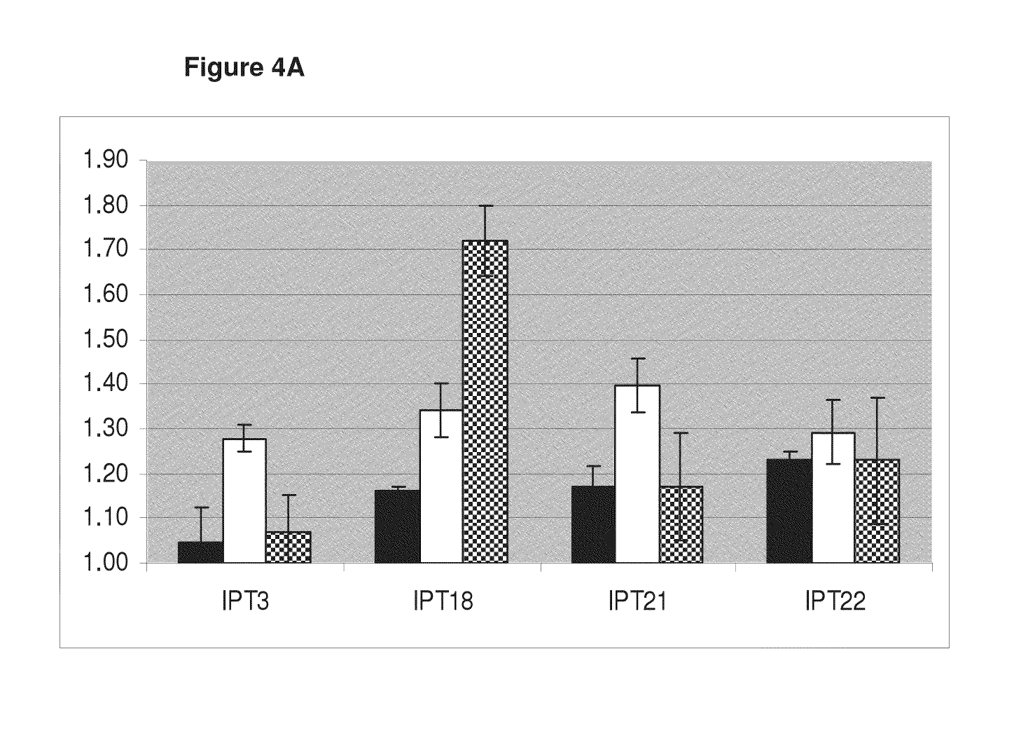

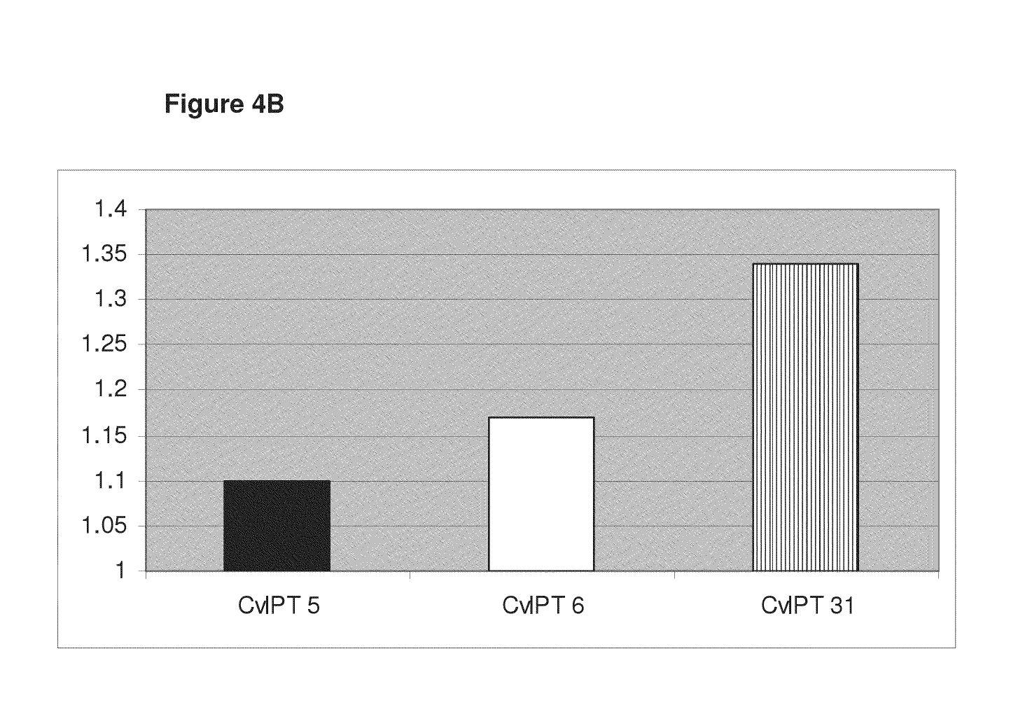

Oil content in the transgenic clones of Chlamydomonas reinhardtii and Chlorella vulgaris was analyzed using Nile Red staining as described above in example 3. Values are normalized against the control. Results of the oil content analysis are detailed tables 5.1 and 5.2 below and in FIGS. 4A-4B.

TABLE 5.1
Relative oil content in transgenic clones
of Chlamydomonas reinhardtii

| | 10 ml | 50 ml | 500 ml |
|---|---|---|---|
| Clone | medium | medium | medium |
| IPT3 | 1.04 ± 0.08 | 1.28 ± 0.03 | 1.07 ± 0.08 |
| IPT18 | 1.16 ± 0.01 | 1.34 ± 0.06 | 1.72 ± 0.08 |
| IPT21 | 1.17 ± 0.05 | 1.40 ± 0.06 | 1.17 ± 0.12 |
| IPT22 | 1.23 ± 0.02 | 1.29 ± 0.07 | 1.23 ± 0.14 |

TABLE 5.2
Relative oil content in transgenic clones of Chlorella vulgaris

| | 50 ml |
|---|---|
| Clone | medium |
| CvIPT5 | 1.10 ± 0.05 |
| CvIPT6 | 1.17 ± 0.02 |
| CvIPT31 | 1.34 ± 0.02 |

As apparent from tables 5.1 and 5.2, and from FIGS. 4A-4B, oil content in the transgenic lines of Chlamydomonas reinhardtii and Chlorella vulgaris, transformed with the IPT gene (SEQ ID NO: 1) was enhanced in comparison with the corresponding control clones.

### Example 5

### Validation of Increased Oil Content in Modified Algal Strains Expressing the IPT Gene (SEQ ID NO: 1)

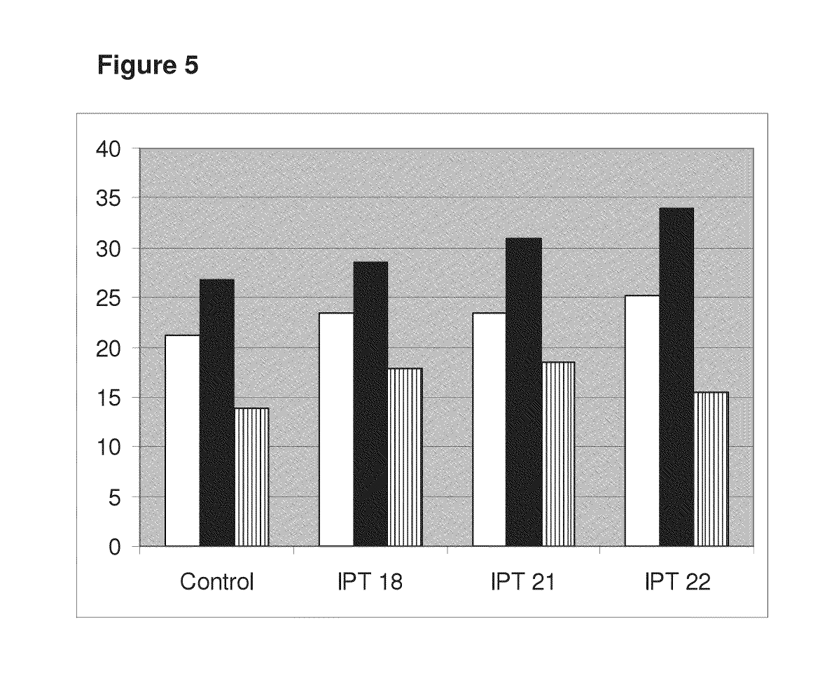

Three transgenic lines of Chlamydomonas reinhardtii, IPT18, IPT21 and IPT22, were grown on a large scale to validate the increase in their oil contents. The algal lines were grown in 1 liter of TAP media for two weeks at 22° C., after which they were harvested and dried for two days at 65° C. The dry algae were sent to the Mylnefield Lipid Analysis laboratory (Dundee, Scotland), where absolute oil content was measured by the gravimetric analysis method (Folch J, Lees M, Sloane Stanley G H (1957) J Biol Chem 226:497-509). The experiment was performed three times in three separate biological repeats, with corresponding measurements of the oil content in the wild type control. The results are presented in table 6 and FIG. 5.

TABLE 6
Oil content in algal lines as determined
by the gravimetric analysis method

| | Oil content | | | | | Increase in |
|---|---|---|---|---|---|---|
| | (% of dry weight) | | | | | oil content |
| Algal | repeat | repeat | repeat | P | | compared to |
| line | 1 | 2 | 3 | value | average | control (%) |
| Control | 21.2 | 26.8 | 13.8 | NA | 20.6 | 0 |
| IPT 18 | 23.4 | 28.5 | 17.8 | 0.014 | 23.2 | 12.6 |
| IPT 21 | 23.4 | 30.9 | 18.5 | 0.029 | 24.3 | 17.9 |
| IPT 22 | 25.1 | 34 | 15.5 | 0.02 | 24.8 | 20.3 |

The enhanced oil content in transgenic lines of Chlamydomonas reinhardtii, grown on a large scale and measured by the gravimetric analysis method, is demonstrated in table 6 and in FIG. 5. These results are in accordance with the oil content values presented in example 4 (table 5.1 and in FIG. 4A), thereby validating the small scale/Nile red staining method of oil content measurement.

The foregoing description of the specific embodiments so fully reveals the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1. A transgenic alga comprising an exogenous transgene, wherein expression of said transgene results in enhanced phytohormone activity in the transgenic alga, and wherein the enhanced phytohormone activity results in increased oil content in said transgenic alga as compared to a corresponding wild type alga.

2. The transgenic alga of claim 1, wherein the enhanced phytohormone activity is of a phytohormone selected from the group consisting of Auxin, Cytokinin, Abscisic acid (ABA) and gibberellic acid (GA₃).

3. The transgenic alga of claim 2, wherein the Auxin is selected from the group consisting of indole-3-acetic acid (IAA), 1-naphthaleneacetic acid (NAA) and 2,4-dichlorophenoxyacetic acid (2,4-D).

4. The transgenic alga of claim 2, wherein the Cytokinin is selected from the group consisting of kinetin, zeatin and 6-benzylaminopurine (6-BAP).

5. The transgenic alga of claim 1, wherein the alga is selected from the group consisting of Green Algae and Diatoms.

6. The transgenic alga of claim 5, wherein the Green Algae is selected from the group consisting of Chlamydomonas reinhardtii, Chlorella vulgaris and Haematococcus pluvialis.

7. The transgenic alga of claim 5, wherein the Diatom is Phaeodactylum tricornutum.

8. The transgenic alga of claim 1, wherein:
(a) the exogenous transgene comprises SEQ ID NO. 1, a fragment thereof, a nucleic acid sequence capable of hybridizing thereto under stringent conditions, or sequences having at least 80% identity thereto; and
(b) the phytohormone which activity is enhanced is Cytokynin.

9. The transgenic alga of claim 1, wherein:
(a) the exogenous transgene comprises a polynucleotide encoding a polypeptide having at least 80% sequence identity to a polypeptide having an amino acid sequence of SEQ ID NO: 4, wherein said encoded polypeptide is a functional homolog of said polypeptide having an amino acid sequence of SEQ ID NO: 4; and
(b) the phytohormone which activity is enhanced is Cytokynin.

10. The transgenic alga of claim 1, wherein the exogenous transgene is introduced into the alga within a genetic construct further comprising a constitutive promoter or an inducible promoter.

11. The transgenic alga of claim 10, wherein the constitutive promoter is pGenD.

12. A method for generating an alga having increased oil content as compared to a control alga, the method comprising enhancing the phytohormone activity of said alga.

13. The method of claim 12, wherein the enhanced phytohormone activity is of a phytohormone selected from the group consisting of Auxin, Cytokinin, Abscisic acid (ABA) and gibberellic acid (GA₃).

14. The method of claim 13, wherein the Auxin is selected from the group consisting of indole-3-acetic acid (IAA), 1-naphthaleneacetic acid (NAA), 2,4-dichlorophenoxyacetic acid (2,4-D).

15. The method of claim 13, wherein the Cytokinin is selected from the group consisting of kinetin, zeatin and 6-benzylaminopurine (6-BAP).

16. The method of claim 12 wherein the enhanced phytohormone activity is achieved by transgenic modification of the alga.

17. The method of claim 16, wherein the transgenic modification comprises incorporation of an exogenous transgene into the alga, and wherein expression of the transgene results in enhanced phytohormone activity.

18. The method of claim 17, wherein:
(a) the exogenous transgene comprises SEQ ID NO. 1, a fragment thereof, a nucleic acid sequence capable of hybridizing thereto under stringent conditions, or sequences having at least 80% identity thereto; and
(b) the phytohormone which activity is enhanced is Cytokynin.

19. The method of claim 17, wherein:
(a) the exogenous transgene comprises a polynucleotide encoding a polypeptide having at least 80% sequence identity to a polypeptide having an amino acid sequence of SEQ ID NO: 4, wherein said encoded polypeptide is a functional homolog of said polypeptide having an amino acid sequence of SEQ ID NO: 4; and
(b) the phytohormone which activity is enhanced is Cytokynin.

20. The method of claim 17, wherein the exogenous transgene is introduced into the alga within a genetic construct further comprising a constitutive promoter or an inducible promoter.

21. The method of claim 20, wherein the constitutive promoter is pGenD.

22. The method of claim 20, wherein the construct further comprises a selectable marker.

23. The method of claim 22, wherein the selectable marker is paromomycin.

24. The method of claim 12, wherein expression of the transgene in the transgenic alga is identified by a method comprising RT-PCR.

25. The method of claim 24, wherein the RT-PCR comprises use of any or both of a forward primer and a reverse primer.

26. The method of claim 25, wherein the forward primer comprises SEQ ID NO. 2.

27. The method of claim 25, wherein the reverse primer comprises SEQ ID NO. 3.

28. The method of claim 12 wherein the enhanced phytohormone activity is achieved by application of phytohormone to the growth medium of the alga.

29. The method of claim 12, wherein the alga is selected from the group consisting of Green Algae and Diatoms.

30. The method of claim 29, wherein the Green Algae is selected from the group consisting of Chlamydomonas reinhardtii, Chlorella vulgaris and Haematococcus pluvialis.

31. The method of claim 29, wherein the Diatom is Phaeodactylum tricornutum.


SEQUENCE LISTING (4 sequences)

SEQ ID NO: 1 - DNA, 732 bp, Agrobacterium tumefaciens
catatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggaattc

SEQ ID NO: 2 - DNA, 20 bp, artificial sequence
  Synthetic
taggctgatcgaggaggtgt

SEQ ID NO: 3 - DNA, 20 bp, artificial sequence
  Synthetic
ctgcttaactctggccttgg

SEQ ID NO: 4 - protein, 240 aa, Agrobacterium tumefaciens
MDLHLIFGPTCTGKTTTAIALAQQTGLPVLSLDRVQCCPQLSTGSGRPTVEELKGTTRLYLDDRPLVEGIIAAKQAHHRLIEEVYNHEANGGLILEGGSTSLLNCMARNSYWSADFRWHIIRHKLPDQETFMKAAKARVKQMLHPAAGHSIIQELVYLWNEPRLRPILKEIDGYRYAMLFASQNQITADMLLQLDANMEGKLINGIAQEYFIHARQQEQKFPQVNAAAFDGFEGHPFGMY